(19) United States
(12) Patent Application Publication
(10) Pub. No.: US 2026/0171257 A1
(43) Pub. Date: Jun. 18, 2026

(54) SYSTEMS AND METHODS FOR MULTI-MODAL GENETIC, BIOMETRIC, AND PSYCHOMETRIC COMPATIBILITY SCORING

(71) Applicant: Deepak Gulati, New York, NY (US)

(72) Inventor: Deepak Gulati, New York, NY (US)

(21) Appl. No.: 19/407,993

(22) Filed: Dec. 3, 2025

Related U.S. Application Data
(60) Provisional application No. 63/734,157, filed on Dec. 15, 2024.

(51) Int. Cl. G16H 50/30; G16B 30/00; G16B 40/00; G16H 10/60; G16H 15/00; G16H 20/00; G16H 50/70

(52) U.S. Cl. CPC G16H 50/30; G16B 30/00; G16B 40/00; G16H 10/60; G16H 15/00; G16H 20/00; G16H 50/70

(57) ABSTRACT

The invention provides systems and methods for computing interpersonal compatibility using multi-modal data fusion that integrates genomic, immunologic, psychometric, biometric, contextual, and reproductive information. Genomic analysis includes variant calling, polygenic scoring, carrier-status evaluation, and HLA/KIR immune-compatibility modeling. Biometric inputs from wearable devices are processed to determine emotional synchrony and autonomic co-regulation. Psychometric and behavioral data are converted into latent-trait embeddings. A machine-learning fusion engine combines all modality-specific feature vectors to generate unified compatibility embeddings and dual outputs representing soulmate-stage suitability and family-planning compatibility. Additional embodiments include donor and surrogate matching, offspring-risk simulation, embryo-viability prediction, and longitudinal model refinement using real-world relational, biometric, or reproductive outcomes.

### FIELD OF THE INVENTION

The present invention relates to computational systems and methods for determining interpersonal compatibility between two or more individuals. More specifically, the invention pertains to multi-modal compatibility scoring frameworks that integrate genomic, immunologic, biometric, psychometric, behavioral, contextual, and reproductive-health data through machine-learning and signal-processing techniques. The systems and methods disclosed herein generate composite compatibility scores for relationship suitability, emotional synchrony, donor—recipient matching, surrogate—intended parent matching, and optimized family-planning outcomes. The invention further relates to artificial-intelligence-driven prediction engines, data-fusion architectures, and biologically informed risk-assessment tools designed to provide actionable insight into interpersonal, reproductive, and long-term relational compatibility.

### BACKGROUND OF THE INVENTION

Human compatibility—whether romantic, social, emotional, or reproductive—is influenced by a complex interplay of biological, psychological, genetic, behavioral, and environmental factors. Historically, compatibility assessments have relied primarily on subjective questionnaires, personality tests, or demographic similarity. Such approaches lack biological resolution, are prone to bias, and fail to predict long-term relational outcomes or reproductive risks.

Scientific research has demonstrated that genetic variation, immunologic similarity or dissimilarity, behavioral traits, autonomic synchrony, and contextual factors contribute significantly to interpersonal dynamics. However, existing systems fail to integrate these multi-modal signals into a unified predictive model. No prior technology combines genomic sequencing data, polygenic risk scoring, HLA/KIR compatibility, biometric emotional-synchrony profiling, psychometric embedding, reproductive-risk modeling, and machine-learning fusion architectures into a single evaluation framework.

Existing matchmaking or compatibility-scoring platforms operate without biological input and rely on surveys or simple heuristics. They do not incorporate genomic markers, do not compute polygenic behavioral scores, do not evaluate reproductive compatibility or donor/surrogate suitability, and do not measure emotional or physiological synchrony.

Existing medical-genetic tools assess disease risk or carrier status but are not integrated with psychological or relational prediction frameworks. Psychometric tools measure personality but lack biological grounding. Neither provides a unified compatibility prediction.

The absence of integrated systems results in fragmented decision-making, reduced predictive accuracy, and limited insight for individuals seeking romantic compatibility or optimized reproductive planning. There remains a need for an integrated, AI-driven, multi-omic compatibility system capable of generating:
Relationship quality prediction
Emotional synchrony and bonding potential
“Soulmate-stage” compatibility score
“Family-planning” reproductive health risk score
Offspring phenotype simulations
Genetic safety alerts
Donor and surrogate compatibility matching
Embryo viability prediction
   No such system exists.

Accordingly, the present invention provides systems and methods for multi-modal interpersonal DNA compatibility scoring, donor/surrogate matching, emotional synchrony analysis, reproductive-risk modeling, and biologically informed partner-selection decision support.

### PRIOR ART

Numerous systems exist for assessing interpersonal compatibility, but none integrate multi-modal biological, psychometric, biometric, contextual, and reproductive data into a unified AI-driven scoring architecture. Existing dating systems rely primarily on demographic heuristics or survey-based matching.

Psychological assessments such as Myers-Briggs or the Big Five offer insights but do not incorporate biological, genomic, or reproductive factors. They rely on self-reported traits and cannot model underlying biological drivers.

Genetic screening tools identify disease risk but do not incorporate psychometric data, biometric synchrony, emotional co-regulation, or contextual relational factors.

Studies exploring HLA-based mate selection do not constitute compatibility systems and do not integrate multiple biological modalities.

Wearable-sensor systems monitor physiological signals but do not compute interpersonal emotional synchrony or integrate these metrics with genomic or psychometric data.

Machine-learning frameworks predicting relationship outcomes rely mostly on communication patterns or survey data and lack multi-modal biological inputs.

No known prior art teaches or suggests combining genomic sequencing, HLA/KIR matching, polygenic scoring, psychogenetic modeling, biometric emotional synchrony, contextual metadata, and reproductive-risk analysis within a multi-layer machine-learning fusion engine.

### SUMMARY OF THE INVENTION

The present invention provides systems and methods for determining interpersonal compatibility using a unified, multi-modal AI architecture that integrates genomic, immunologic, psychometric, biometric, contextual, and reproductive-health data.

In one embodiment, the system includes a genomic subsystem that performs variant calling, carrier-risk analysis, polygenic scoring, and immune-compatibility modeling.

In another embodiment, a biometric subsystem processes physiological signals from wearable sensors and computes emotional synchrony and physiologic co-regulation metrics.

A psychogenetic subsystem produces latent-trait embeddings combining psychometric inputs with behavioral-genetic markers.

A machine-learning fusion engine integrates all subsystem outputs into a multi-modal compatibility embedding.

A dual-mode scoring engine outputs a soulmate-stage compatibility score and a family-planning compatibility score.

Additional embodiments include donor matching, surrogate matching, embryo-viability prediction, reproductive-risk modeling, and reinforcement-learning updates in real time from wearable sensors during interactions from real-world outcomes.

### BRIEF DESCRIPTION OF THE DRAWINGS

FIG. 1 illustrates the multi-modal compatibility system architecture.

FIG. 1A illustrates the feature-extraction pipeline.

FIG. 2 illustrates the genomic analysis pipeline.

FIG. 3 illustrates the biometric emotional-synchrony engine.

FIG. 4 illustrates the reproductive-analysis engine.

FIG. 5 illustrates the dynamic-weighting engine.

FIG. 6 illustrates the donor-matching engine.

FIG. 7 illustrates the surrogate-matching engine.

FIG. 8 illustrates the emotional-synchrony processor.

FIG. 9 illustrates the psychogenetic modeling engine.

FIG. 10 illustrates the embryo-viability prediction engine.

FIG. 11 illustrates the population compatibility graph.

FIG. 12 illustrates the closed-loop learning engine.

FIG. 13 illustrates the dual acquisition pipelines.

### DETAILED DESCRIPTION OF THE INVENTION

The following detailed description enables any person skilled in the art to make and use the invention. The embodiments described herein are illustrative and not limiting. Variations, substitutions, and modifications may be made without departing from the scope of the invention as defined by the claims.

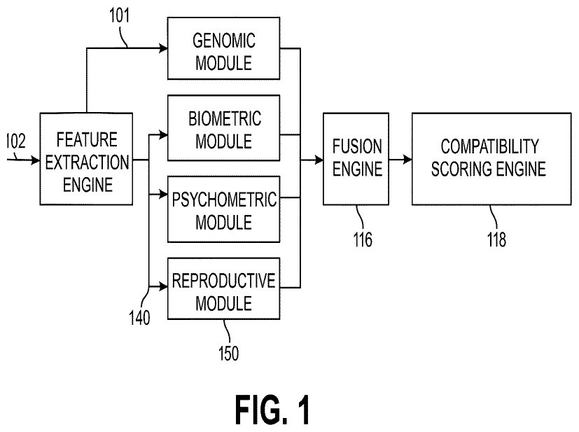

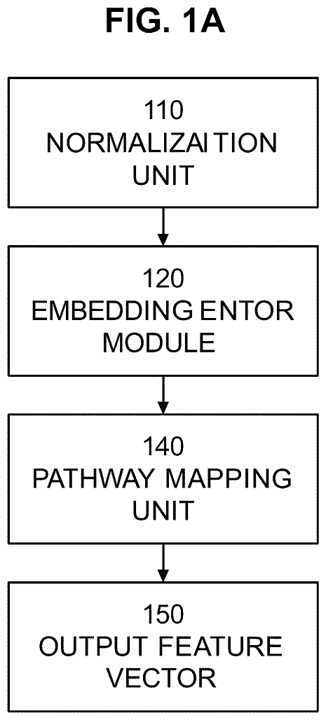

### System Overview (FIG. 1)

FIG. 1 illustrates system 100 for generating multi-modal interpersonal compatibility scores. System 100 includes one or more computing devices configured to receive, store, process, and integrate genomic data, psychometric data, biometric data, contextual data, and reproductive-health information.

Raw data are processed through modular subsystems that produce structured feature vectors. A machine-learning fusion engine combines these vectors into a unified compatibility embedding from which multiple compatibility scores are derived.

In various embodiments, users provide input through a mobile application, wearable device, web interface, or integrated laboratory or data provider. The system receives raw data, performs multi-stage preprocessing and feature engineering, and outputs compatibility scores, risk assessments, donor suitability rankings, surrogate suitability rankings, and predictive insights regarding relational or reproductive outcomes.

### Feature Extraction Subsystem (FIG. 1A)

FIG. 1A illustrates feature-extraction subsystem 110. Raw data from genomic sources, psychometric questionnaires, wearable-sensor streams, or contextual metadata are first directed to Normalization Unit 110.

Normalization Unit 110 performs data standardization and scaling using statistical normalization, z-score transformation, thresholding, trimming, and domain-specific preprocessing strategies.

Normalized data are passed to Embedding Generator 120, which converts categorical, numerical, and structured biological markers into continuous embedding vectors using neural encoders, PCA, autoencoders, transformers, or specialized feature-hashing.

Embedding outputs are then processed by Event Detection Module 130, which identifies discrete or periodic events of biological or behavioral relevance, including autonomic activations, variant clusters, or psychometric latent-trait transitions.

Pathway Mapping Unit 140 maps detected events to biological, psychological, or contextual pathways based on known associations.

Output Feature Vector 150 aggregates these processed features into a consolidated representation for downstream fusion.

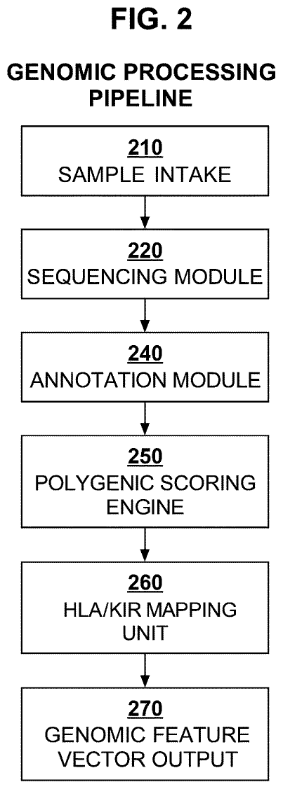

### Genomic Processing Pipeline (FIG. 2)

FIG. 2 illustrates genomic-processing pipeline 200. Sample Intake 210 receives DNA data from sequencing providers or genotyping arrays.

Sequencing Module 220 may operate internally or through laboratory APIs. Variant Calling Unit 230 identifies single nucleotide variants, insertions, deletions, structural variants, and copy-number variations.

Annotation Module 240 annotates variants using public databases and proprietary datasets.

Polygenic Scoring Engine 250 computes polygenic risk scores for behavioral traits, personality traits, disease predispositions, and reproductive risks.

HLA/KIR Mapping Unit 260 computes immunologic compatibility between individuals.

Genomic Feature Vector Output 270 provides the structured genomic representation used for downstream analysis.

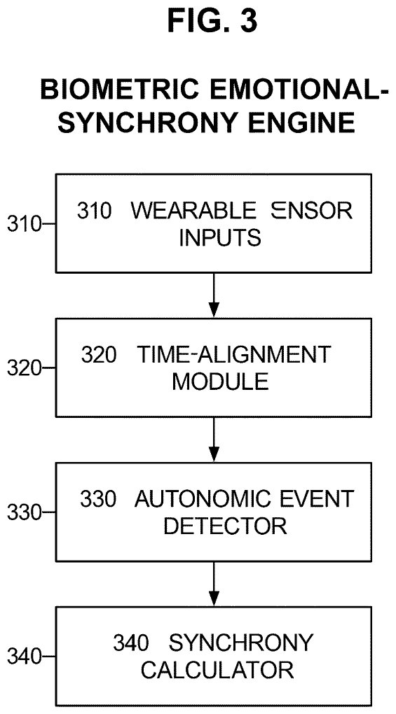

### Biometric Emotional-Synchrony Engine (FIG. 3)

FIG. 3 illustrates biometric subsystem 300. Wearable Sensor Inputs 310 include physiological measurements such as heart rate, heart-rate variability, skin conductance, respiration, accelerometry, movement patterns, temperature, and peripheral blood flow.

Time-Alignment Module 320 synchronizes physiological data streams between two individuals using cross-correlation, temporal alignment, or dynamic time warping.

Autonomic Event Detector 330 identifies shared physiological events, including co-occurring peaks, dips, and autonomic activations.

Synchrony Calculator 340 computes inter-individual resonance metrics such as correlation, coherence, co-regulation, phase alignment, and shared autonomic patterns.

Biometric Synchrony Score 350 represents the physiological synchrony between individuals.

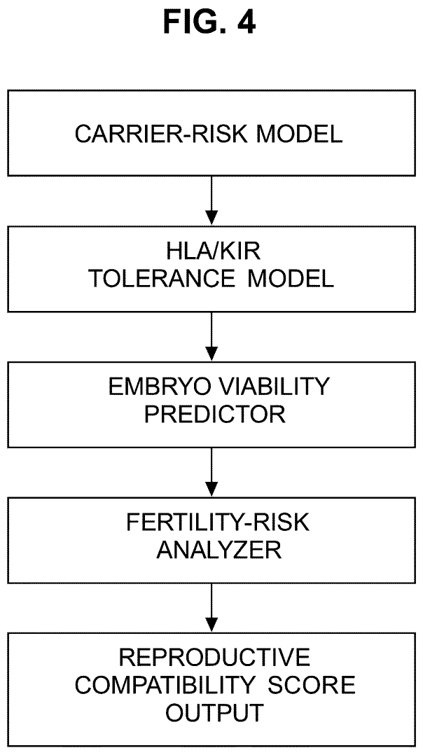

### Family-Planning Compatibility Engine (FIG. 4)

FIG. 4 illustrates reproductive subsystem 400. Carrier-Risk Model 410 evaluates combined recessive and X-linked carrier status.

HLA/KIR Tolerance Model 420 computes immunologic reproductive compatibility.

Embryo Viability Predictor 430 integrates parental genomic information, polygenic embryo simulation, chromosomal-stability metrics, and aneuploidy likelihood.

Fertility-Risk Analyzer 440 models reproductive risk intersections including infertility, miscarriage susceptibility, or congenital-disease risk.

Reproductive Compatibility Score Output 450 provides the family-planning compatibility estimation.

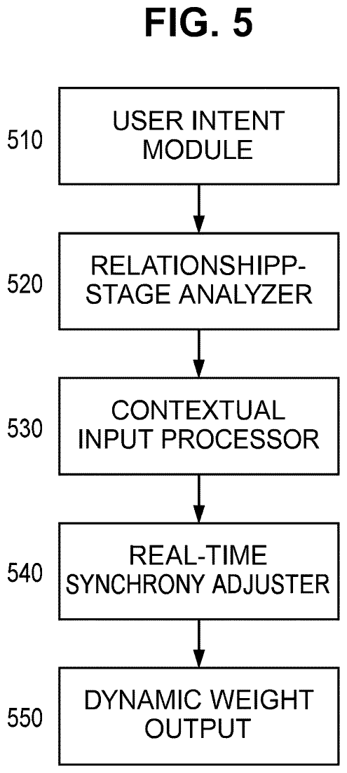

### Dynamic Weighting Engine (FIG. 5)

FIG. 5 illustrates subsystem 500 for dynamic weighting. Inputs include User Intent Module 510, Relationship-Stage Analyzer 520, Contextual Input Processor 530, and Real-Time Synchrony Adjuster 540.

These modules modify feature importance in real time based on relationship goals, emotional resonance, temporal context, and observed synchrony.

Dynamic Weight Output 550 adjusts modality-specific weights used by the fusion engine.

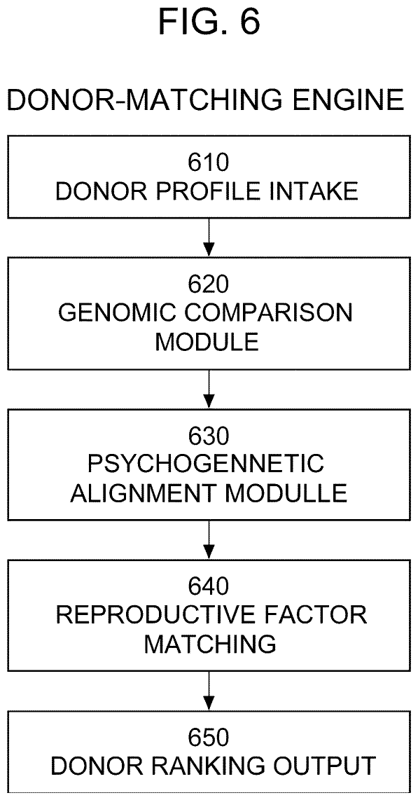

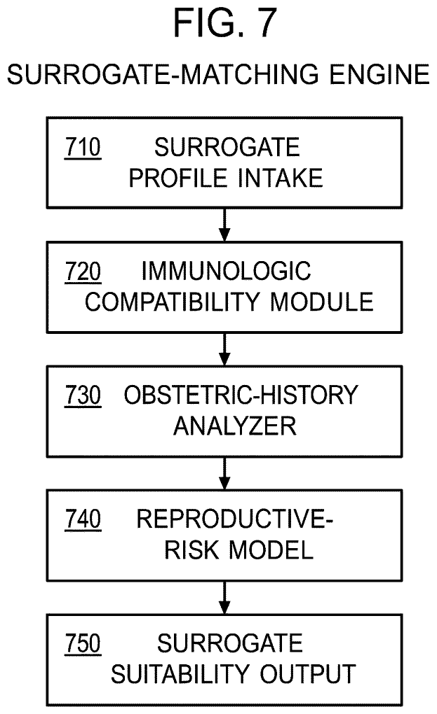

### Donor and Surrogate Matching Engines (FIG. 6 & FIG. 7)

### Donor Matching (FIG. 6)

FIG. 6 illustrates donor-matching engine 600.

Donor Profile Intake 610 collects donor identity, medical data, and genetic profiles.

Genomic Comparison 620 evaluates genetic compatibility.

Psychogenetic Alignment 630 evaluates behavioral and psychological alignment.

Reproductive Factor Matching 640 evaluates reproductive compatibility.

Donor Ranking Output 650 produces a ranked suitability list.

### Surrogate Matching (FIG. 7)

FIG. 7 illustrates surrogate-matching engine 700.

Surrogate Profile Intake 710 collects surrogate data.

Immunologic Compatibility Module 720 evaluates HLA/KIR immunologic alignment.

Obstetric-History Analyzer 730 evaluates prior pregnancy and delivery history.

Reproductive-Risk Model 740 evaluates risks related to gestation.

Surrogate Suitability Output 750 produces a surrogate-matching ranking.

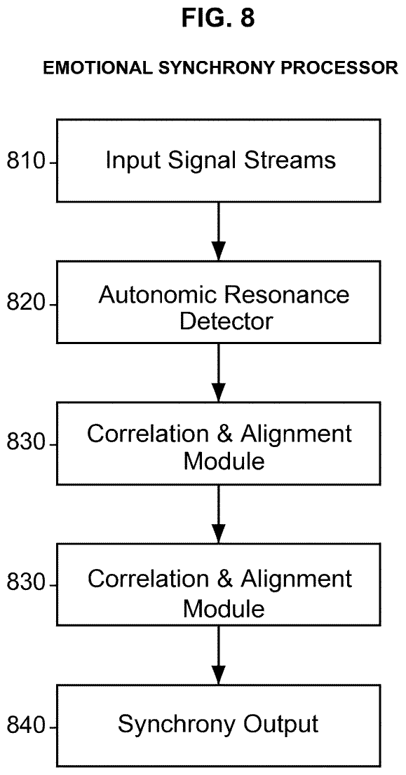

### Emotional Synchrony Processing (FIG. 8)

FIG. 8 illustrates emotional-synchrony processor 800.

Input Signal Streams 810 are received from wearable sensors.

Autonomic Resonance Detector 820 identifies co-regulated events.

Correlation and Alignment Module 830 computes synchrony metrics.

Synchrony Output 840 provides a multimodal emotional synchrony score.

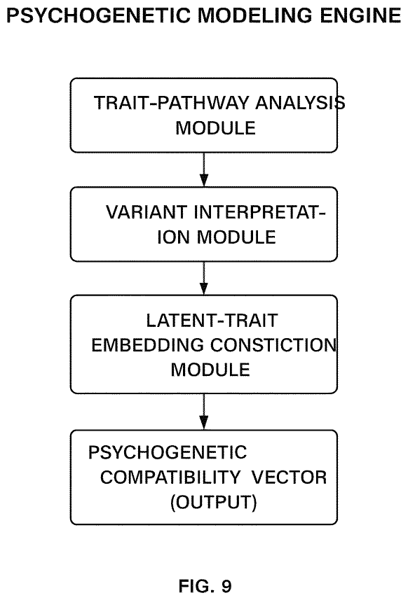

### Psychogenetic Modeling (FIG. 9)

FIG. 9 illustrates psychogenetic modeling engine 900.

Behavioral-Gene Inputs 910 include genetic markers associated with behavioral traits.

Psychometric Assessment Module 920 processes questionnaire responses.

Latent-Trait Embedding Generator 930 produces multidimensional embeddings.

Psychogenetic Scoring Module 940 computes alignment across traits.

Compatibility Output 950 provides the psychogenetic compatibility score.

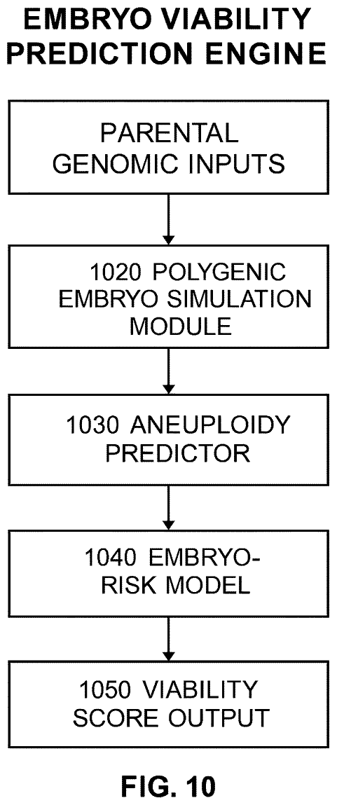

### Embryo Viability Prediction (FIG. 10)

FIG. 10 illustrates embryo-viability engine 1000.

Parental Genomic Inputs 1010 provide genetic background.

Polygenic Embryo Simulation 1020 models likely embryo genotypes.

Aneuploidy Predictor 1030 estimates chromosomal-stability risk.

Embryo-Risk Model 1040 integrates risk metrics.

Viability Score Output 1050 ranks embryo viability.

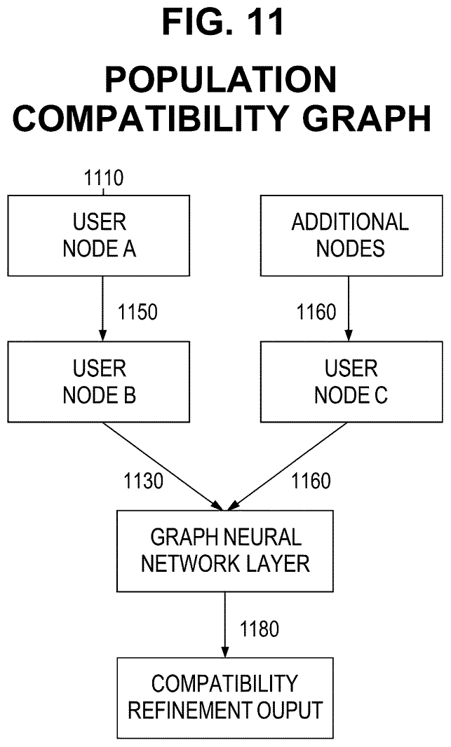

### Population Compatibility Graph (FIG. 11)

FIG. 11 shows population graph 1100 with nodes representing individuals and edges weighted by compatibility scores.

Graph Neural Network Layer 1170 refines pairwise scores using population-level structure.

Refinement Output 1180 strengthens compatibility predictions over time.

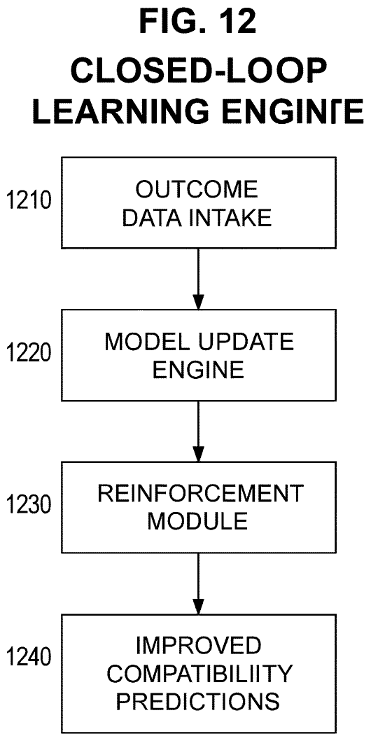

### Closed-Loop Learning Engine (FIG. 12)

FIG. 12 illustrates a closed-loop learning engine 1200.

Outcome Intake 1210 receives real-world relationship or reproductive outcomes.

Model Update Engine 1220 retrains underlying models.

Reinforcement Module 1230 updates compatibility scoring parameters.

Improved Predictions 1240 enhance model accuracy.

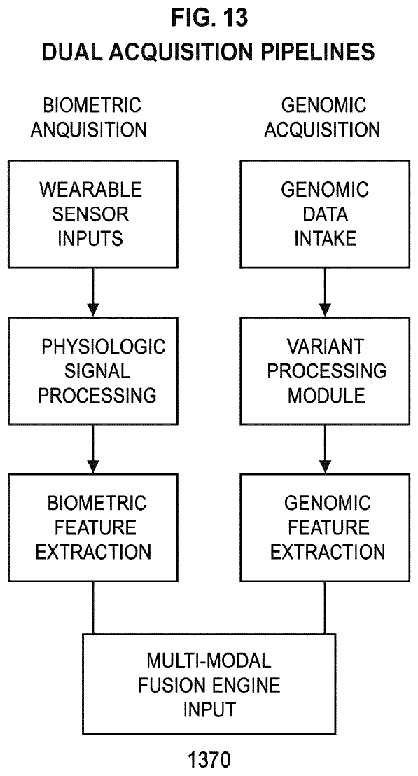

### Dual Acquisition Pipelines (FIG. 13)

FIG. 13 illustrates two parallel pipelines.

Biometric Pipeline 1310-1330 processes wearable data.

Genomic Pipeline 1340-1360 processes DNA data.

Fusion Engine Input 1370 integrates both streams for compatibility scoring.

1. A system for determining interpersonal compatibility between two individuals, comprising:
a genomic analysis module configured to receive DNA sequence data from each individual and generate genomic feature vectors;
a biometric processing module configured to receive physiologic signals from wearable sensors and compute biometric synchrony values;
a psychogenetic modeling module configured to generate latent-trait embeddings based on psychometric assessments and behavioral-genetic markers;
a reproductive-analysis module configured to compute family-planning compatibility parameters including carrier-risk intersections, embryo-viability predictors, and HLA/KIR tolerance metrics;
a machine-learning fusion engine configured to combine the genomic feature vectors, biometric synchrony values, latent-trait embeddings, and reproductive-compatibility parameters into a multi-modal compatibility embedding; and
a dual-mode scoring engine configured to output a soulmate-stage compatibility score and a family-planning-stage compatibility score.

2. The system of claim 1, wherein the soulmate-stage compatibility score comprises weighted contributions from genomic similarity, immunologic complementarity, psychogenetic alignment, emotional synchrony, and contextual relationship factors.

3. The system of claim 1, wherein the family-planning-stage compatibility score comprises weighted contributions from carrier-risk intersections, reproductive polygenic predictions, HLA/KIR tolerance modeling, embryo-viability predictions, and long-term reproductive outcome estimates.

4. The system of claim 1, wherein the genomic analysis module is further configured to compute polygenic risk scores across a plurality of health-related, behavioral, reproductive, and personality-associated traits.

5. The system of claim 1, wherein the biometric processing module is configured to perform time alignment, autonomic-event detection, and multi-signal correlation to generate emotional-synchrony metrics.

6. The system of claim 1, wherein the psychogenetic modeling module comprises a neural-network encoder configured to generate latent-trait embeddings from psychometric inputs, behavioral-gene inputs, and contextual-trait metadata.

7. The system of claim 1, wherein the reproductive-analysis module is configured to generate embryo-viability predictions using polygenic embryo simulation and chromosomal-stability estimation.

8. The system of claim 1, further comprising a donor-matching engine configured to compute donor-recipient compatibility based on genomic, psychogenetic, biometric, and reproductive-health attributes.

9. The system of claim 1, further comprising a surrogate-matching engine configured to compute surrogate-recipient compatibility using immunologic compatibility scores, obstetric-history analysis, and reproductive-risk modeling.

10. The system of claim 1, wherein the machine-learning fusion engine comprises a multi-layer neural network configured to adjust modality-specific weights based on user intent, relationship stage, and real-time synchrony signals.

11. A computer-implemented method for determining interpersonal compatibility, comprising:
receiving genomic data from two individuals;
generating genomic feature vectors;
receiving wearable-sensor data;
generating biometric synchrony values;
receiving psychometric responses;
generating latent-trait embeddings;
computing reproductive-compatibility parameters;
fusing all data modalities using a machine-learning model; and
outputting at least one compatibility score.

12. The method of claim 11, further comprising constructing a population-scale compatibility graph comprising nodes representing individuals and edges representing pairwise compatibility weights.

13. The method of claim 11, wherein generating biometric synchrony values comprises computing autonomic co-regulation, phase-alignment, and shared-event synchrony metrics.

14. The method of claim 11, wherein generating latent-trait embeddings comprises encoding psychometric inputs and behavioral-gene markers using a neural-network encoder.

15. The method of claim 11, further comprising computing embryo-viability predictions using polygenic embryo simulation.

16. The method of claim 11, further comprising computing reproductive-risk intersections based on combined carrier-status data.

17. The method of claim 11, further comprising computing contextual-compatibility attributes including lifestyle, environmental, geographic, and historical relationship factors.

18. The method of claim 11, wherein fusing the data modalities comprises applying a multi-modal transformer, graph neural network, recurrent neural network, or ensemble thereof.

19. The method of claim 11, further comprising outputting a donor-matching score.

20. The method of claim 11, further comprising outputting a surrogate-matching score.

21. The system of claim 1, wherein the fusion engine includes a contextual-weighting mechanism configured to adjust scoring parameters based on situational data.

22. The system of claim 1, wherein emotional synchrony is weighted more heavily during soulmate-stage scoring and reproductive compatibility is weighted more heavily during family-planning-stage scoring.

23. The method of claim 11, further comprising adjusting modality-specific weights based on relationship stage.

24. The method of claim 11, wherein donor-matching incorporates psychogenetic alignment between donor and intended parent.

25. The method of claim 11, wherein surrogate-matching incorporates immunologic tolerability parameters.

26. The system of claim 1, wherein the psychogenetic modeling module generates a dual-trait embedding comprising behavioral-genetic inputs and psychometric inputs.

27. The method of claim 11, further comprising applying reinforcement learning to update compatibility predictions based on real-world outcomes.

28. The system of claim 1, further comprising a closed-loop learning engine configured to retrain model parameters based on relationship success, emotional-synchrony performance, fertility outcomes, or donor/surrogate match satisfaction.

29. The system of claim 1, wherein the compatibility embedding is refined using a population-level graph neural network.

30. The method of claim 11, further comprising generating predictive probabilities of long-term relational stability.

31. The method of claim 11, further comprising computing HLA/KIR complementarity values.

32. The system of claim 1, wherein the reproductive-analysis module evaluates risk for recessive conditions.

33. The system of claim 1, wherein embryo-viability prediction incorporates polygenic trait modeling.

34. The method of claim 11, further comprising generating implantation-likelihood estimates for simulated embryos.

35. The system of claim 1, wherein the machine-learning fusion engine incorporates reproductive-health history features.

36. The method of claim 11, wherein outputting compatibility further comprises generating immunologic-risk alerts.

37. The system of claim 1, further comprising a module configured to evaluate immune-system tolerance between individuals.

38. The method of claim 11, wherein computing family-planning compatibility comprises integrating monogenic, polygenic, immunologic, and chromosomal-stability factors.

39. The system of claim 1, wherein the reproductive-analysis module includes a gestational-outcome predictor.

40. The method of claim 11, further comprising modeling offspring genetic projections.

41. The system of claim 1, wherein emotional synchrony comprises correlation between autonomic-event sequences.

42. The method of claim 11, further comprising calculating synchrony-decay rates or resonance-duration metrics.

43. The system of claim 1, wherein biometric data include heart rate, skin conductance, respiratory cycles, motion patterns, and temperature.

44. The system of claim 1, wherein the psychogenetic module merges behavioral-gene markers with psychometric inputs using a multi-modal encoder.

45. The method of claim 11, further comprising computing reproductive polygenic-risk factors for each individual.

46. The system of claim 1, wherein embryo-viability predictions incorporate inheritance-pattern simulation.

47. The method of claim 11, wherein psychogenetic scoring comprises mapping traits to a continuous compatibility manifold.

48. The system of claim 1, wherein reinforcement-learning modules adjust psychogenetic weighting factors.

49. The method of claim 11, further comprising generating sperm-genomic and egg-genomic compatibility vectors.

50. The system of claim 1, wherein contextual inputs include lifestyle, environmental, socioeconomic, circadian-rhythm, and geographic metadata.

51. The method of claim 11, further comprising generating compatibility-based match recommendations.

52. The system of claim 1, wherein compatibility scores are stored in an encrypted database.

53. The method of claim 11, wherein outputting compatibility comprises generating multi-dimensional compatibility reports.

54. The system of claim 1, wherein donor-matching and surrogate-matching share a unified multi-modal compatibility framework.

55. The method of claim 11, further comprising ranking a plurality of donor candidates.

56. The method of claim 11, further comprising ranking a plurality of surrogate candidates.

57. The system of claim 1, wherein dynamic weighting adapts based on user-selected relationship goals.

58. The system of claim 1, wherein compatibility embeddings are stored for repeated inference.

59. The method of claim 11, further comprising computing multi-party compatibility among groups of three or more individuals.

60. The system of claim 1, wherein the dual-mode scoring engine outputs a combined compatibility index representing both soulmate-stage and family-planning compatibility.
